(19) United States
(12) Patent Application Publication
(10) Pub. No.: US 2012/0280189 A1
(43) Pub. Date: Nov. 8, 2012

(54) PERFLUOROKETONES AS GASEOUS DIELECTRICS

(76) Inventors: Karl J. Warren, Hudson, WI (US); Phillip E. Tuma, Faribault, MN (US); John G. Owens, Woodbury, MN (US); Richard M. Minday, Stillwater, MN (US)

(21) Appl. No.: 13/260,994

(22) PCT Filed: Jan. 19, 2011
(86) PCT No.: PCT/US2011/021659

§ 371 Date: Sep. 29, 2011

Related U.S. Application Data
(60) Provisional application No. 61/297,991, filed on Jan. 25, 2010.

(51) Int. Cl. H01B 3/56; H01F 27/20; H01B 7/42; C07C 49/00

(52) U.S. Cl. 252/571; 568/303; 336/55; 174/15.6

(57) ABSTRACT

A gaseous dielectric comprising a perfluoroketone of the formula R_{f}¹—CO—R_{f}², wherein each of R_{f}¹ and R_{f}² are perfluoroaliphatic groups, and use thereof in electrical devices, is described.

### CROSS REFERENCE TO RELATED APPLICATION

This application claims the benefit of U.S. Provisional Patent Application No. 61/297,991, filed Jan. 25, 2010, the disclosure of which is incorporated by reference herein in its entirety.

### FIELD OF THE INVENTION

This invention relates to perfluoroketones and the use thereof as gaseous dielectric fluids in electrical devices such as capacitors, switchgear, transformers and electric cables or buses.

### BACKGROUND

Dielectric gases are used in various electrical apparatus; see for example U.S. 2008/0135817 (Luly et al.). Major types of such apparatus are transformers, electric cables or buses, and circuit breakers or switchgear. In such electrical devices, dielectric gases are often used in place of air due to their high dielectric constant (K) and high dielectric strength (DS). Such dielectric gases allow higher power densities as compared to air-filled electrical devices.

Most significantly sulfur hexafluoride (SF₆) has become the dominant captive dielectric gas in many electrical applications. SF₆ is advantageously nontoxic, non-flammable, easy to handle, has a useful operating temperature range, and excellent dielectric and arc-interrupting properties. Within transformers, it also acts as a coolant. Blowers within the transformer circulate the gas aiding in heat transfer from the windings.

However, the greatest concern with SF₆ is its 3200 year atmospheric lifetime and very significant global warming potential (GWP) of about 22,200 times the global warming potential of carbon dioxide. At the December 1997 Kyoto Summit in Japan, representatives from 160 countries drafted an agreement containing limits for greenhouse gas emissions. The agreement covers six gases, including SF₆, and includes a commitment to lower the total emissions of these gases by the year 2010 to levels 5.2% below their total emissions in 1990. See UNEP (United Nations Environment Programme), Kyoto Protocol to the United Nations Framework Convention on Climate Change, Nairobi, Kenya, 1997.

The National Institute of Standards and Technology (NIST) have published Technical Note 1425: “Gases for electrical Insulation and Arc Interruption: Possible Present and Future Alternatives to Pure SF₆”, which identifies, as possible replacements, mixtures of SF₆ with either nitrogen or helium, or high-pressure nitrogen. Some other replacement mixtures suffer from release of free carbon during arcing, increased toxicity during or after arcing, and increased difficulty in gas handling due to substantially different pressures required during liquification of the components. Also identified are perfluorocarbon (PFC) gases that might also be mixed with nitrogen or helium, like SF₆. Yet PFCs also have high GWPs so the possible reduction in environmental impact of such strategies is limited.

### SUMMARY

Briefly, the present disclosure provides a gaseous dielectric comprising a perfluoroketone of the formula R_{f}¹—CO—R_{f}², wherein each of R_{f}¹ and R_{f}² are perfluoroaliphatic groups. The gaseous dielectric may be useful in a number of other applications that use dielectric gases. Examples of such other applications are described in the aforementioned NIST technical note 1425. The disclosure further provides an electrical device containing as a component the perfluoroketone gaseous dielectric. In some embodiments, the present disclosure further provides a gaseous dielectric comprising a mixture of a perfluoroketone and an inert gas, such as nitrogen.

The use of a perfluoroketone as a gaseous dielectric advantageously has a broad range of operating temperatures and pressures, is thermally, and chemically stable, has a higher dielectric strength and heat transfer efficiency than SF₆ at a given partial pressure, and has and a lower global warming potential (GWP) than SF₆. The instant perfluoroketones generally have a dielectric strength greater than 6 kV at a pressure of 20 kPa at the operating temperature of the electrical device.

### BRIEF DESCRIPTION OF THE FIGURES

FIG. 1 is a graph of the heat transfer performance of the gaseous perfluoroketone/nitrogen dielectrics as compared to SF₆, and SF₆ mixtures with N₂, at the indicated pressures.

FIG. 2 is a graph of the dielectric strength performance of the gaseous perfluoroketone dielectrics as compared to SF₆.

FIG. 3 is an illustration of electrical hardware using a perfluoroketone gaseous dielectric.

As used herein, “GWP” is a relative measure of the warming potential of a compound based on the structure of the compound. The GWP of a compound, as defined by the Intergovernmental Panel on Climate Change (IPCC) in 1990 and updated in 2007, is calculated as the warming due to the release of 1 kilogram of a compound relative to the warming due to the release of 1 kilogram of CO₂ over a specified integration time horizon (ITH).

$${GWP}_{i} ( {t}^{′} ) = \frac{{\int }_{0}^{ITH} {a}_{i} [ C ( t ) ] d t}{{\int }_{0}^{ITH} {a}_{{CO}_{2}} [ {C}_{{CO}_{2}} ( t ) ] d t} = \frac{{\int }_{0}^{ITH} {a}_{i} {C}_{oi} {e}^{- t \text{/} π} d t}{{\int }_{0}^{ITH} {a}_{{CO}_{2}} [ {C}_{{CO}_{2}} ( t ) ] d t}$$

In this equation αᵢ is the radiative forcing per unit mass increase of a compound in the atmosphere (the change in the flux of radiation through the atmosphere due to the IR absorbance of that compound), C is the atmospheric concentration of a compound, τ is the atmospheric lifetime of a compound, t is time and i is the compound of interest.

The commonly accepted ITH is 100 years representing a compromise between short-term effects (20 years) and longer-term effects (500 years or longer). The concentration of an organic compound, i, in the atmosphere is assumed to follow pseudo first order kinetics (i.e., exponential decay). The concentration of CO₂ over that same time interval incorporates a more complex model for the exchange and removal of CO₂ from the atmosphere (the Bern carbon cycle model).

Carbonyl compounds such as aldehydes and ketones have been shown to have measurable photolysis rates in the lower atmosphere resulting in very short atmospheric lifetimes. Compounds such as formaldehyde, acetaldehyde, propionaldehyde, isobutyraldehyde, n-butyraldehyde, acetone, 2-butanone, 2-pentanone and 3-pentanone have atmospheric lifetimes by photolysis ranging from 4 hours to 38 days (Martinez, R. D., et al., 1992, Atmospheric Environment, 26, 785-792, and Seinfeld, J. H. and Pandis, S. N., Atmospheric Chemistry and Physics, John Wiley & Sons, New York, p. 288, 1998). CF₃CF₂C(O)CF(CF₃)₂ has an atmospheric lifetime of approximately one week based on photolysis studies with natural sunlight (D'Anna, B., Sellevag, S. R., Wirtz, K., Nielsen, C. J., Environ. Sci. Technol., 39, 8708, 2005), and photolysis studies at 300 nm are described by Taniguchi, N., et al. J. Phys. Chem. A, 107(15), 2674-79, 2003. Other perfluoroketones show similar absorbances near 300 nm and are expected to have similar atmospheric lifetimes.

The very short lifetimes of the perfluoroketones lead to very low GWPs. A measured IR cross-section was used to calculate the radiative forcing value for CF₃CF₂C(O)CF(CF₃)₂ using the method of Pinnock, et al. (J. Geophys. Res., 100, 23227, 1995). Using this radiative forcing value and the one week atmospheric lifetime the GWP (100 year ITH) for CF₃CF₂C(O)CF(CF₃)₂ is 1. The perfluoroketones of the disclosure typically have a GWP less than about 100, and preferably less than 10.

As a result of their rapid degradation in the lower atmosphere, the perfluoroketones have short lifetimes and would not be expected to contribute significantly to global warming. The low GWP of the perfluoroketones, in addition to the dielectric performance characteristics, make them well suited for use as a gaseous dielectric.

Advantageously, the gaseous dielectric of the present disclosure has a high electrical strength, also described as high breakdown voltage. “Breakdown voltage,” as used in this application means (at a specific frequency) the highest voltage applied to a liquid that induces catastrophic failure of the gaseous dielectric allowing electrical current to conduct through the gas. Thus the gaseous dielectric of the present invention can function under high voltages. The gaseous dielectric can also exhibit a low loss factor, that is, the amount of electrical energy that is lost as heat from an electrical device such as a capacitor.

Perfluoroketones (PFKs) that are useful in the present invention include those ketones having only fluorine attached to the carbon backbone. More specifically, the instant perfluoroketones are of the formula R_{f}¹—CO—R_{f}², wherein each of R_{f}¹ and R_{f}² are perfluoroaliphatic groups, preferably perfluoroalkyl groups. The perfluoroketones contain 4 to 7 carbon atoms.

More specifically, R_{f}¹ and R_{f}² are each monovalent perfluoroaliphatic groups having 1 to 5 perfluorinated carbon atoms, optionally containing one or more catenary (in-chain) heteroatoms, such as divalent oxygen, hexavalent sulfur, or trivalent nitrogen bonded only to carbon atoms, such heteroatoms being a chemically stable link between perfluorocarbon portions of the perfluoroaliphatic group and do not interfere with the inert character of the perfluoroaliphatic group. In preferred embodiments, R_{f}¹ and R_{f}² are perfluoroalkyl groups. The skeletal chain of R_{f}¹ and R_{f}² can be straight chain, branched chain, and if sufficiently large, cyclic, or combinations thereof, such as perfluoroalkylcycloaliphatic groups. In some embodiments at least one of R_{f}¹ and R_{f}² is a branched perfluoraliphatic group.

“Perfluoroaliphatic” is inclusive of perfluoroalkyl and perfluorooxyalkyl (and nitrogen and sulfur analogs thereof) wherein all hydrogen atoms of the oxyalkyl radical are replaced by fluorine atoms and the number of carbon atoms is from 2 to 5, e.g. CF₃CF₂OCF₂CF₂—, CF₃CF₂SF₄CF₂— or CF₃CF₂N(CF₃)CF₂—.

“Perfluoroalkyl” has essentially the meaning as “alkyl” wherein all of the hydrogen atoms of the alkyl radical are replaced by fluorine atoms and the number of carbon atoms is from 1 to about 5, e.g. perfluoropropyl, perfluoroisopropyl perfluorobutyl, perfluoromethyl, and the like.

Perfluorinated ketones (PFKs) useful in the present invention include ketones which are fully fluorinated, i.e., all of the hydrogen atoms in the carbon backbone have been replaced with fluorine atoms. The carbon backbone can be linear, branched, or cyclic, or combinations thereof, and will preferably have about 4 to about 7 carbon atoms. Representative examples of perfluorinated ketone compounds suitable for use in the processes and compositions of the invention include CF₃CF₂C(O)CF(CF₃)₂, CF₃C(O)C₂F₅, CF₃C(O)(CF₂)₂CF₃, CF₃CF₂C(O)CF₂CF₂CF₃, (CF₃)₂CFC(O)CF(CF₃)₂, CF₃(CF₂)₂C(O)CF(CF₃)₂, CF₃(CF₂)₄C(O)CF₃, CF₃(CF₂)₃C(O)CF₃, CF₃CF₂C(O)CF₂CF₃, CF₃C(O)CF(CF₃)₂, perfluorocyclopentanone, perfluorocyclohexanone, and mixtures thereof.

The perfluoroketones can also contain one or more caternary (i.e. in-chain) heteroatoms interrupting the carbon backbone. Suitable heteroatoms include, for example, nitrogen, oxygen, and sulfur atoms. Representative examples of such fluorinated ketones include CF₃OCF₂CF₂C(O)CF(CF₃)₂ and CF₃OCF₂C(O)CF(CF₃)₂.

In addition to demonstrating dielectric gas performance, perfluorinated ketones can offer additional important benefits in safety of use and in environmental properties. For example, CF₃CF₂C(O)CF(CF₃)₂ has low acute toxicity, based on short-term inhalation tests with mice exposed for four hours at a concentration of 100,000 ppm in air. Also based on photolysis studies at 300 nm CF₃CF₂C(O)CF(CF₃)₂ has an estimated atmospheric lifetime of one week. Other perfluorinated ketones show similar absorbances and thus are expected to have similar atmospheric lifetimes. As a result of their rapid degradation in the lower atmosphere, the perfluorinated ketones have short atmospheric lifetimes and would not be expected to contribute significantly to global warming (i.e., low global warming potentials) and thereby reduce greenhouse gas emissions when replacing SF₆.

Perfluorinated ketones which are straight chain or cyclic can be prepared as described in U.S. Pat. No. 5,466,877 (Moore et al.) which in turn can be derived from the fluorinated esters described in U.S. Pat. No. 5,399,718 (Costello et al.).

Perfluorinated ketones that are alpha-branched to the carbonyl group can be prepared as described in U.S. Pat. No. 3,185,734 (Fawcett et al.). Hexafluoropropylene is added to acyl halides in an anhydrous environment in the presence of fluoride ion. Small amounts of hexafluoropropylene dimer and/or trimer impurities can be removed by distillation from the perfluoroketone. If the boiling points are too close for fractional distillation, the dimer and/or trimer impurity can be removed by oxidation with alkali metal permanganate in a suitable organic solvent such as acetone, acetic acid, or a mixture thereof. The oxidation reaction is typically carried out in a sealed reactor at ambient or elevated temperatures. In some embodiments, perfluoroketones in which at least one of R_{f}¹ or R_{f}² are secondary perfluoroalkyl groups are preferred.

Linear perfluorinated ketones can be prepared by reacting a perfluorocarboxylic acid alkali metal salt with a perfluorocarbonyl acid fluoride as described in U.S. Pat. No. 4,136,121 (Martini et al.) Such ketones can also be prepared by reacting a perfluorocarboxylic acid salt with a perfluorinated acid anhydride in an aprotic solvent at elevated temperatures as described in U.S. Pat. No. 5,998,671 (Van Der Puy). All of the aforementioned patents are incorporated by reference in their entirety.

The useful perfluoroketones have a gaseous range that encompasses the operating temperature range of the electrical device in which they are used as components of the gaseous dielectric of this invention, preferably such that the perfluoroketones have a boiling point less than 50° C., more preferably below 30° C. and containing 4 to 7 carbon atoms. C₃ perfluoroketone, i.e. hexafluoroacetone, may be excluded due to the known toxicity—having a Threshold Limit Value of 0.1 ppm. Higher, i.e. greater than C₇, perfluoroketones may be excluded due to the low vapor pressure.

In most embodiments, useful perfluoroketones have a vapor pressure of 30 kPa at 25° C. Preferably, useful perfluoroketones have a vapor pressure of at least 30 kPa, more preferably at least 40 kPa, at the operating temperature of the electrical device. Generally, useful perfluoroketone gaseous dielectrics having a boiling point in the range of −20 to 50° C., preferably −20 to 30° C. For example, many electrical devices such as capacitors, transformers, circuit breakers and gas insulated transmission lines may operate at temperatures of at least 30° C. and above. At these operating temperatures, the gaseous dielectric should have a vapor pressure of at least 40 kPa.

Further, the perfluoroketones have a dielectric strength of at least 5 kV at the operating pressure in the electric device, which is typically at least 20 kPa. Preferably perfluoroketones have a dielectric strength of at least 10 kV and more preferably at least 15 kV at the operating temperature and pressure of the device.

In some embodiments, the perfluoroketone may be combined with other conventional gaseous dielectrics, such as an inert gas. These conventional dielectric gases have a boiling points below 0° C., have a zero ozone depletion potential, a global warming potential below that of SF₆ (about 22,000), are chemically and thermally stable, and have a dielectric constant greater than air. The conventional gaseous dielectrics include nitrogen, helium, argon, and carbon dioxide. Generally, the second gaseous dielectric is used in amounts such that vapor pressure is at least 70 kPa at 25° C., or at the operating temperature of the electrical device. In some embodiments the ratio of the vapor pressure of the second gaseous dielectric to the perfluoroketone dielectric is at least 2.5:1, preferably at least 5:1, and more preferably at least 10:1.

The perfluoroketones are useful in gaseous phase for electrical insulation and for arc quenching and current interruption equipment used in the transmission and distribution of electrical energy. Generally, there are three major types of electrical devices in which the gases of the present disclosure can be used: (1) gas-insulated circuit breakers and current-interruption equipment, (2) gas-insulated transmission lines, and (3) gas-insulated transformers. Gas-insulated substations contain one or all of these devices often in fluid communication with each other. Such gas-insulated equipment is a major component of power transmission and distribution systems all over the world.

In some embodiments, the present disclosure provides electrical devices, such as capacitors, comprising metal electrodes spaced from each other such that the gaseous dielectric fills the space between the electrodes. The interior space of the electrical device may also comprise a reservoir of the liquid perfluoroketone which is in equilibrium with the gaseous perfluoroketone. Thus the reservoir may replenish any losses of the gaseous perfluoroketone.

For circuit breakers the excellent thermal conductivity and high dielectric strength of such gases, along with the fast thermal and dielectric recovery (short time constant for increase in resistivity), are the main reasons for its high interruption capability. These properties enable the gas to make a rapid transition between the conducting (arc plasma) and the dielectric state of the arc, and to withstand the rise of the recovery voltage.

For gas-insulated transformers the heat transfer performance, and compatibility with current devices, in addition to the dielectric characteristics, make them a desirable medium for use in this type of electrical equipment. The instant perfluoroketones have distinct advantages over oil insulation, including none of the fire safety problems or environmental compatibility, high reliability, little maintenance, long service life, low toxicity, ease of handling, and reduced equipment weight.

For gas-insulated transmission lines the dielectric strength of the gaseous perfluoroketones under industrial conditions is significant, especially the behavior of the gaseous dielectric under metallic particle contamination, switching and lightning impulses, and fast transient electrical stresses. These gaseous perfluoroketones also have a high efficiency for transfer of heat from the conductor to the enclosure and are stable for long periods of time (e.g., 40 years). These gas-insulated transmission lines offer distinct advantages: cost effectiveness, high-carrying capacity, low losses, availability at all voltage ratings, no fire risk, reliability, and a compact alternative to overhead high voltage transmission lines in congested areas that avoids public concerns with overhead transmission lines.

For gas-insulated substations, the entire substation (circuit breakers, disconnects, grounding switches, busbar, transformers, etc., are interconnected) is insulated with the gaseous dielectric medium of the present disclosure, and, thus, all of the above-mentioned properties of the dielectric gas are significant.

In some embodiments the gaseous dielectric may be present in an electric device as a gas per se, or as a gas in equilibrium with the liquid. In these embodiments the liquid phase serves as a reservoir for additional gaseous dielectric.

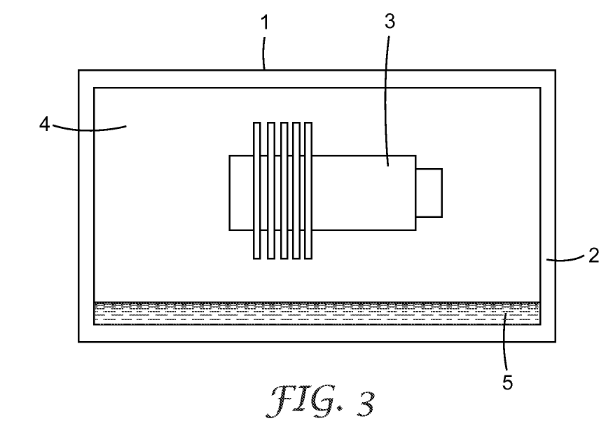

The use of perfluoroketones as gaseous dielectrics is illustrated in the generic electrical device of FIG. 3. The Figure illustrates device comprising a tank or pressure vessel 2, containing electrical hardware 3, such as a switch, interrupter or the windings of a transformer, and at least one gaseous perfluoroketone 4. Optionally the gaseous perfluoroketone 4 is in equilibrium with a reservoir of a liquid perfluoroketone 5.

Objects and advantages of this invention are further illustrated by the following examples, but the particular materials and amounts thereof recited in these examples, as well as other conditions and details, should not be construed to unduly limit this invention.

### EXAMPLES

### Materials

| Compound | Structure | Name | Source |
|---|---|---|---|
| C5K | i-C₃F₇CF(O)CF₃ | 1,1,1,3,4,4,4- | Prep 1 |
| | | heptafluoro-3- | |
| | | trifluoromethyl- | |
| | | butan-2-one | |
| C6K | i-C₃F₇C(O)C₂F₅ | 1,1,1,2,2,4,5,5,5- | 3M Company, |
| | | nonafluoro-4- | St. Paul, MN |
| | | trifluoromethyl- | |
| | | pentan-3-one | |
| SF₆ | SF₆ (99.5% purity) | Sulfur | Concorde Gas, |
| | | hexafluoride | Eatontown, NJ |

### Preparation 1: 1,1,1,3,4,4,4-Heptafluoro-3-trifluoromethyl-butan-2-one CF₃C(O)CF(CF₃)₂

Trifluoroacetic anhydride (2310 g 11.0 mol, Alfa Aesar, Ward Hill, Mass.), potassium fluoride (703 g 12.1 mol, Aldrich, Milwaukee, Wis.), hexafluoropropene (1650 g 11.0 mol, MDA Manufacturing, Decatur, Ala.) and diglyme solvent (2000 g) were combined in a 2-gallon Parr high pressure reactor. The reactor was then heated slowly to 75° C. The pressure increased to 350 psi. As the hexafluoropropene reacted with the anhydride to form the ketone, the pressure gradually dropped below 50 psi. Additional hexafluoropropene was added at this point (528 g) to maximize conversion. The reaction mix was stirred for 24 hours. The crude reaction product was emptied to a round bottom flask where the ketone product was vacuum distilled away from the salts and diglyme solvent. The ketone was then purified by fractional distillation from concentrated sulfuric acid (used for drying). An Oldershaw (20-tray) column was used for the distillation. The total amount of ketone recovered was 1690 g. Product purity was measured by ¹H, ¹⁹F NMR and determined to be 99.6%.

### Heat Transfer Measurements

The relative heat transfer capabilities of SF₆, SF₆/N₂ mixtures and C6K-saturated N₂ were measured experimentally using the following apparatus. The apparatus comprised a 1 liter jacketed pressure vessel. The pressure vessel contained an electric resistance heater and a DC fan, and a valved pressure inlet for introduction of gases and purging of the chamber. Water of a controlled temperature, T_{w}, is passed through the jacket. Thermocouples are used to monitor the heater temperature, Tₕ, the water temperature and the temperatures of the gas in the vessel, Tₐ. The vessel was first evacuated. The gas under study is then added. In the case of SF₆ and SF₆/N₂ measurements, the composition of the gas is controlled by first adding SF₆ until a particular pressure is obtained, P_{gas}. For 100% SF₆ this is the total pressure, Pₜₒₜ. For compositions below 100% SF₆, N₂ is then added to obtain Pₜₒₜ. The composition can then be calculated as Vol %=P_{gas}/Pₜₒₜ.

For C6K, it is not possible for P_{gas} to exceed the C6K saturation pressure, Pₛₐₜ, at Tₐ. P_{gas} will equal Pₛₐₜ=EXP(−3627.0355/Ta+22.7598) if excess liquid is present. Therefore, the gas was added as a liquid beyond the level that would saturate the vessel volume at the maximum temperature to be studied. N₂ was added to obtain the desired Pₜₒₜ. Power is then applied to the heater and fan. Data are recorded when the system has equilibrated. For these experiments water temperatures were 14.9, 30.9, and 43.4° C. at Pₜₒₜ=4 atm and 15.7, 31.0 and 42.6, at Pₜₒₜ=6 atm.

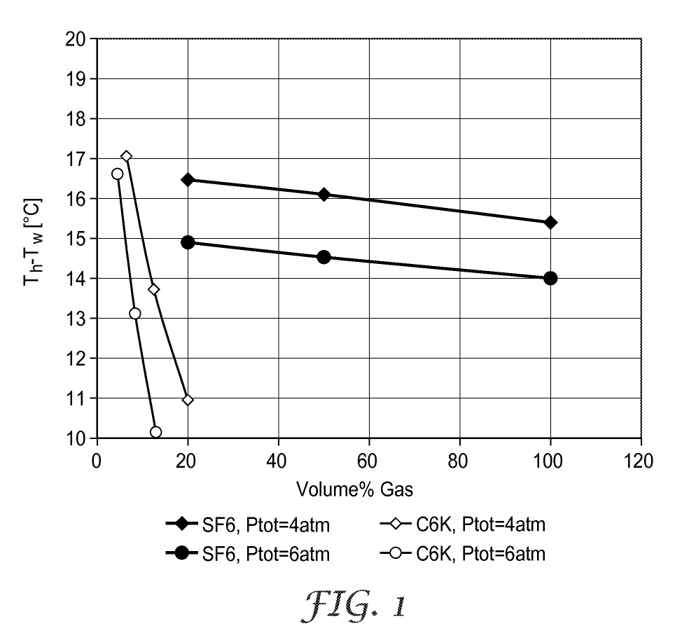

Superior heat transfer performance is indicated by a lower temperature difference between the jacket water temperature, T_{w}, and the heater temperature, Tₕ, at a given Pₜₒₜ and, in the case of the C6K, T_{w}. The data in FIG. 1 show that at even at moderate gas temperatures, C6K-saturated N₂ produced superior heat transfer performance as compared to pure SF₆.

### Dielectric Strength (DS) Measurement

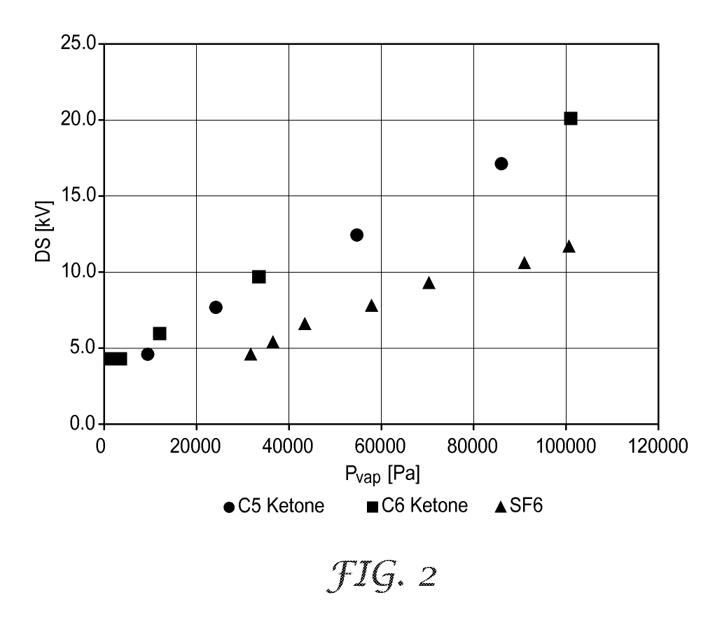

The dielectric strength of SF₆, C5K and C6K were measured experimentally using a dielectric Hipotronics OC90D dielectric strength tester (available from Hipotronics, Brewster, N.Y.) modified to allow low pressure gases. The electrode and test configuration comply with ASTM D877. The test chamber was first evacuated and the baseline dielectric strength was measured. Known quantities of SF₆, C6K or C5K were then injected to achieve the measured pressure, Pᵥₐₚ. The dielectric strength (DS) was recorded after each injection. The results are shown in FIG. 2.

1. An electrical device containing as a component a C₄ to C₇ perfluoroketone gaseous dielectric, wherein the perfluoroketone has a vapor pressure of at least 30 kPa at the operating temperature of the device.

2. The electrical device of claim 1 comprising a perfluoroketone of the formula R_{f}¹—CO—R_{f}², wherein each of R_{f}¹ and R_{f}² are perfluoroaliphatic groups.

3. The electrical device of claim 1 wherein the perfluoroketone is a C₄ to C₆ perfluoroketone.

4. The electrical device of claim 1 wherein said perfluoroketone is selected from the group consisting of CF₃CF₂C(O)CF(CF₃)₂, CF₃C(O)C₂F₅, CF₃C(O)(CF₂)₂CF₃, CF₃CF₂C(O)CF₂CF₂CF₃, (CF₃)₂CFC(O)CF(CF₃)₂, CF₃(CF₂)₂C(O)CF(CF₃)₂, CF₃(CF₂)₄C(O)CF₃, CF₃(CF₂)₃C(O)CF₃, CF₃CF₂C(O)CF₂CF₃, CF₃C(O)CF(CF₃)₂, perfluorocyclopentanone, perfluorocyclohexanone, and mixtures thereof.

5. The electrical device of claim 1 wherein said perfluoroketone is selected from CF₃CF₂C(O)CF(CF₃)₂ and CF₃C(O)CF(CF₃)₂.

6. The electrical device of claim 1 wherein one of R_{f}¹ and R_{f}² of the gaseous dielectric is a secondary perfluoroalkyl.

7. The gaseous dielectric of claim 1 having a vapor pressure of at least 30 kPa at 25° C.

8. The gaseous dielectric of claim 1 having a dielectric strength of at least 5 kV at 25 kPa.

9. The electrical device of claim 1 wherein the gaseous dielectric has a global warming potential of less than 100.

10. The electrical device of claim 1 wherein the gaseous dielectric further comprises a reservoir of liquid dielectric perfluoroketone.

11. The electrical device of claim 1 wherein the gaseous dielectric has a dielectric strength (DS) of 6 kV of more.

12. The electrical device of claim 1 wherein the gaseous dielectric has a global warming potential of less than 10.

13. The electrical device of claim 1 wherein at least one of R_{f}¹ or R_{f}² is (CF₃)₂CF—.

14. The electrical device of claim 1, wherein said electrical device is selected from the group consisting of: gas-insulated circuit breakers and current-interruption equipment, gas-insulated transmission lines, gas-insulated transformers, and gas-insulated substations.

15. The electrical device of claim 1 further comprising a second dielectric gas having a vapor pressure of at least 70 kPa.

16. The electrical device of claim 15 wherein the second dielectric gas is selected from nitrogen, helium, argon, and carbon dioxide.

17. A gaseous dielectric composition comprising a C₄ to C₇ perfluoroketone gaseous dielectric, and a second gaseous dielectric comprising an inert gas having a vapor pressure is at least 70 kPa.

18. The gaseous dielectric composition of claim 17 wherein the ratio of the vapor pressure of the second gaseous dielectric to the perfluoroketone dielectric is at least 2.5:1.

19. The gaseous dielectric composition of claim 17 wherein the inert gas is selected from nitrogen, helium, argon, and carbon dioxide.
